(19) United States
(12) Patent Application Publication
(10) Pub. No.: US 2021/0133637 A1
(43) Pub. Date: May 6, 2021

(54) SYSTEM FOR SCHEDULING SUMMONING ASSISTANCE

(71) Applicant: ALEXANDER COVARRUBIAS, Norht Bay Village, FL (US)

(72) Inventor: ALEXANDER COVARRUBIAS, Norht Bay Village, FL (US)

(21) Appl. No.: 16/674,142

(22) Filed: Nov. 5, 2019

(51) Int. Cl. G06Q 10/02; G16H 40/20; G06Q 50/26

(52) U.S. Cl. CPC G06Q 10/02; G06Q 50/26; G16H 40/20

(57) ABSTRACT

A scheduling and summoning system that provides users with specialty services providers that are available and within their geographic location. The system is composed of a central data and processing system, a user interface and a provider interface. The central data and processing system has a data capturing module, a user data module, a provider data module, a background screening module, a provider qualification module, a geographic location module, a provider availability module and a service completion module. The user interface allows a user to communicate with the central data processing system. The provider interface allows the provider to communicate with the central data processing system. The system allows user's requiring a service to be paired with qualified providers that can provide a service.

### TECHNICAL FIELD

The present invention pertains generally to the field of online scheduling and summoning trustworthy assistance via wireless networks.

### BACKGROUND OF THE INVENTION

Home health care networks assist incapacitated patients. The patients are normally referred to the home health care networks via insurance companies, hospitals or governmental agencies. On occasions, family members will contact the networks to contract private care.

The Networks normally contract private nurses or nurse assistants to assist the patients. The insurance companies, the hospitals, the governmental agencies or the family members never have a say of whom will treat the patient or when treatment will take place.

The present invention is directed to a system that will allow someone requesting assistance, medical or not, to select the person whom will provide the assistance and the time that it will be provided.

The person requesting the assistance will have access to a network that will display individual providers whom can provide the assistance requested and whom are within a certain geographical location when the assistance is requested. Individual providers that are not within the geographic area will not be displayed to the person requesting the assistance.

The system ensures that individual providers displayed on their network have been verified to be law abiding individuals that are qualified to work on the individual matters requiring assistance.

The system ensures that the user of the system will only see qualified individual providers that are available within the geographical area of the user when the user requires the assistance.

The system will ensure that the only individual providers trained to provide the assistance requested by the user are displayed to the user.

The system will ensure that users of the system will not see individual providers caring for another user at the time user requires assistance, this feature ensures that users will be cared for in an efficient manner.

The present invention provides persons requesting assistance with a system that will connect them to assistance providers that will serve their needs in a secure, timely, trained and efficient manner.

### SUMMARY OF THE INVENTION

The present invention is directed a scheduling and summoning system that provides users with specialty services providers that are available and that are within the user's geographic location. Specialty service providers shall be defied to be home health care providers, assisted living facilitators or any other qualified individual that cares for others.

The system comprises of a central data and processing system, a user interface and a provider interface. The central data and processing system has a data capturing module, a user data module, a provider data module, a background screening module, a provider qualification module, a geographic location module, a provider availability module and a service completion module. The user interface allows a user to communicate with the central data system. The provider interface allows the provider to communicate with the central data and processing system. The central data and processing system of the present invention allows user's requiring a service to be paired with qualified providers that can provide a service. For the user and the provider to be paired, the qualified provider must be in the same geographic location as the user and cleared for service by the central data system.

The central data and processing system captures all users' information and the providers' information and stores them for future use. The central data and processing system is the driver of scheduling and summoning system of the present invention. It ensures that only providers that meet the need of the user are dispatched to the user when the providers are cleared for service by the central data and processing system and they are in the vicinity of the user.

An object of the present invention is to provide a user with a scheduling and summoning system that will provide a user with a list of providers whom are qualified to provide a service requested.

Another object of the present invention it to provide a system that will allow a user requesting a service with the ability to select a person whom has been background screened to be able to provide the service requested.

Another object of the present invention is to provide a safe feature that allows the present invention to determine when a service to a user has commenced and when the service has terminated.

Yet another object of the present invention is to provide a user with the ability to choose a specific service provider that will render the services to the user.

Yet still another object of the present invention is to provide a fail safe that only displays providers not rendering services to other users to a user requesting a service.

### BRIEF DESCRIPTION OF THE DRAWINGS

These and other features, aspects, and advantages of the present invention will become better understood with regards to the following description, appended claims, and drawings where:

FIG. 1 is a diagram that shows the central data system, the user interface and the provider interface of the present invention; and

FIG. 2 is a diagram that shows the modules of the central data system.

### DETAILED DESCRIPTION OF THE INVENTION

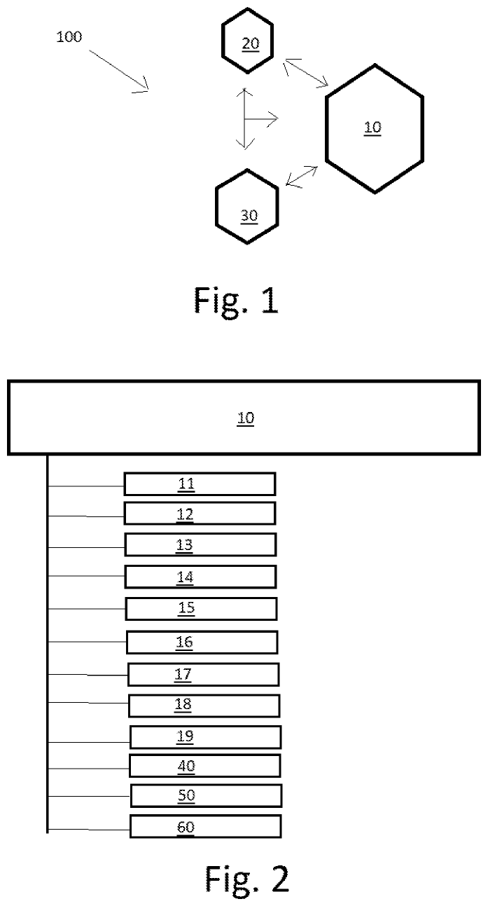

Referring to FIGS. 1-2, the present invention is a scheduling and summoning system 100 that provides users with specialty services providers that are available and within their geographic location. The system comprises of a central data system 10, a user interface 20 that communicates with the central data system 10 and a provider interface 30 that communicates with the central data system 10. The central data system comprises 10: a data capturing module 11 wherein a user can place the data that relates to the services that the user is requesting and the time frame in which the service is needed and a provider can place his or her data or credentials and his or her provider's unique communication device that will be associated with the provider; a user data module 12 that will store the user's data; a provider data module 13 that will store the provider's data; a background screening module 14 that will provide a background screen of the provider to ensure that the provider can render the services requested by the user and that the provider is a law abiding citizen that can provide the service requested, the background screen module will fail providers whom have criminal histories or whom have a mental health related illness that would deem them unsafe to work with the public; a provider qualification module 15 that will only maintain providers passing background screens on the system; a geographic location module 16 that will determine a user's geographic location and a provider's geographic location that will be based on the provider's unique communication device; a provider availability module 17 that will allow any user requesting a service to see any provider on the system that can provide the service requested that is within the user's geographic location; and a service completion module 18 that allows the user to inform the central data system 10 of any detail relating to the service requested and wherein the provider can inform the central data system 10 when the service is commenced and when the service is terminated. The user interface 20 that allows a user to communicate with the central data system 10, wherein the user can place the user's personal data and a service request need into the central data system 10. The provider interface 30 allows the provider to communicate with the central data system 10, wherein the provider can place the provider's data or credentials and wherein the provider can register the provider's unique communication device 30 with the central data system 10.

The provider availability module 17 has a fail-safe that will not display providers providing services to other users when a user's request is made. This feature does not allow providers schedule two tasks simultaneously, thereby ensuring that the user receives the service requested in a time efficient manner.

A further embodiment of the central data and processing system 10 comprises a task timeout module 19, wherein the task timeout module 19 monitors the time from when a provider reaches a user and the providers leaves the user, the task timeout module will not allow that provider to be displayed to any other user until the central data and processing system 10 has cleared the provider for another job.

Still a further embodiment of the present invention, the central data and processing system 10 comprises of a provider rating module 40 that will allow users to rate the provider. The provider rating module 40 allows users to rate providers provided services so that other users can consider the provider prior to selecting the provider.

In Still a further embodiment, the central data and processing system 10 comprises of a user rating module 50 that will allow providers to rate the user and to record the needs of the user and the services provided to the user.

In yet still another embodiment of the present invention, the central data and processing system 10 will comprise of a communication module 60 that will allow the user interface 20 and the provider interface 30 to communicate directly and that will record all communications between the user interface 20 and the provider interface 30.

The user interface 20 of the present invention is used by the user as follows: 1. It allows the user to access the central data and processing system; 2. It allows the user to place all of the pertinent information that relates to the user and his needs into the central data nd processing system; 3. It allows the user to request a service; 4. It allows the user to determine the time in which the service will be rendered; 5. It allows a user to see providers within his or her geographic location that can provide the service; 6. It allows the user to preview the provider whom will provide the service prior to selecting the provider; 7. It will allow the user to rate the provider whom provided the service; 8. It will allow the user to inform the central data and processing system when the service has been completed; and 9. It shall allow the user to communicate directly with the provider in a secured and recorded line.

The provider interface shall be used by the provider in the following manner: 1. It will allow the provider to register into the central data and processing system; 2. It will allow the provider to upload his qualifications into the central data and processing system; 3. It will allow the provider to inform the central data and processing system of the provider's geographic location and of his or her availability of accepting jobs; 4. It will allow the provider to inform the system of job completions and details that correspond to each job completed; and 5. It shall allow the provider to communicate directly with the user in a secured and recorded line.

The central data and processing system of the present invention is programmed to ensure that all modules within the central data and processing system work together to pair a user requiring assistance with a qualified provider that will render the assistance in a time efficient manner.

An advantage of the present invention is that it provides a user with a scheduling and summoning system that provides the user with a list of providers whom are qualified to provide the services requested.

Another advantage of the present invention it that it provides a system that allow a user requesting a service with the ability to select a person whom has been background screened to be able to provide the service requested to provide the service.

Another advantage of the present invention is that it provides a safe feature that allows the present invention to determine when a service to a user has commenced and when the service has terminated.

Yet another advantage of the present invention is that it provides a user with the ability to choose a specific service provider that will render the services to the user.

Yet still another object of the present invention is to provide a fail safe that only displays providers not rendering services to other users to a user requesting a service.

The embodiments of the apparatus and method of use described herein are exemplary and numerous modifications, combinations, variations, and rearrangements can be readily envisioned to achieve an equivalent result, all of which are intended to be embraced within the scope of the appended claims. Further, nothing in the above-provided discussions of the apparatus and method should be construed as limiting the invention to a particular embodiment or combination of embodiments. The scope of the invention is defined by the appended claims.

What is claimed is:

1. A scheduling and summoning system that provides users with specialty services providers that are available and within their geographic location, the system comprises:
a central data and processing system that comprises:
a data capturing module wherein a user can place the data that relates to the services that the user is requesting and the time frame in which the service is needed and a provider can place his or her data or credentials and his or her provider's unique communication device that will be associated with the provider;
a user data module that will store the user's data;
a provider data module that will store the provider's data;
a background screening module that will provide a background screen of the provider to ensure that the provider can render the services requested by the user and that the provider is a law abiding citizen that can provide the service requested, the background screen module will fail providers whom have criminal histories or whom have a mental health related illness that would deem them unsafe to work with the public;
a provider qualification module that will only maintain providers passing background screens on the central data and processing system;
a geographic location module that will determine a user's geographic location and a provider's geographic location that will be based on the provider's unique communication device;
a provider availability module that will allow any user requesting a service to see any provider on the system that can provide the service requested that is within the user's geographic location; and
a service completion module that allows the user to inform the central data and processing system of any detail relating to the service requested and wherein the provider can inform the central data and processing system when the service is commenced and when the service is terminated;
a user interface that allows a user to communicate with the central data and processing system, wherein the user can place the user's personal data and a service request need into the central data and processing system; and
a provider interface that allows the provider to communicate with the central data and processing system, wherein the provider can place the provider's data or credentials and wherein the provider can register the provider's unique communication device with the central data and processing system.

2. The scheduling and summoning system that provides users with specialty services providers that are available and within their geographic location of claim 1, wherein the provider availability module will not display providers providing services to other users when a user's request is made.

3. The scheduling and summoning system that provides users with specialty services providers that are available and within their geographic location of claim 2, the central data and processing system comprises a task timeout module, wherein the task timeout module monitors the time from when a provider reaches a user and the providers leaves the user, the task timeout module will not allow that provider to be displayed to any other user until the central data and processing system has cleared the provider for another job.

4. The scheduling and summoning system that provides users with specialty services providers that are available and within their geographic location of claim 3, the central data and processing system comprises a provider rating module that will allow users to rate the provider.

5. The scheduling and summoning system that provides users with specialty services providers that are available and within their geographic location of claim 4, the central data and processing system comprises a user rating module that will allow providers to rate the user and to record the needs of the user and the services provided to the user.

6. The scheduling and summoning system that provides users with specialty services providers that are available and within their geographic location of claim 5, the central data and processing system comprises a communication module that allow the user interface and the provider interface to communicate directly and that records all communications between the user interface and the provider interface.
